(19) United States
(12) Patent Application Publication
(10) Pub. No.: US 2003/0216317 A1
(43) Pub. Date: Nov. 20, 2003

(54) Protein having cell-recognizing activity and/or cytocidal activity

(75) Inventors: Michio Ohba, Fukuoka-shi (JP); Eiichi Mizuki, Kurume-shi (JP); Tetsuyuki Akao, Chikugo-shi (JP); Hiroyuki Saito, Kurume-shi (JP); Hedeki Katayama, Chikushino-shi (JP); Satoko Yamashita, Kurume-shi (JP); Dae-Weon Lee, College Station, TX (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Fukuoka Prefecture, Fukuoka-shi (JP)

(21) Appl. No.: 10/403,181

(22) Filed: Mar. 28, 2003

(30) Foreign Application Priority Data
Mar. 29, 2002 (JP) 2002-097634

(51) Int. Cl. A61K 38/16; C12Q 1/68; C07K 14/32; C07H 21/04; C12N 1/21; C12N 15/74

(52) U.S. Cl. 514/12; 530/350; 435/6; 435/69.1; 435/252.31; 435/320.1; 536/23.7

(57) ABSTRACT

The present invention provides a protein consisting of the amino acid sequence represented by SEQ ID NO: 2, as well as proteins comprising an amino acid sequence wherein one or more amino acids are deleted, substituted, added or inserted in the amino acid sequence represented by SEQ ID NO: 2, whose activated form has a cell-recognizing activity and/or a cytopathic activity. The present invention also provides a nucleic acid molecule consisting of the nucletide sequence represented by SEQ ID NO: 1, as well as nucleic acid molecules which hybridize under stringent conditions to a nucleic acid molecule having a nucleotide sequence complementary to the nucleotide sequence represented by SEQ ID NO: 1, and which encode a protein having a cell-recognizing activity and/or a cytopathic activity in an activated form.

### CROSS-REFERENCE TO RELATED APPLICATION

The present application claims benefit to the filing date of Japanese patent application No. 2002-097634, filed Mar. 29, 2002, which is herein incorporated by reference.

### TECHNICAL FIELD OF THE INVENTION

The present invention relates to a novel protein derived from bacteria. Specifically, the present invention relates to a novel protein having a cell-recognizing activity and/or a cytocidal activity, and a gene encoding the protein, which are derived from Bacillus thuringiensis (hereinafter optionally abbreviated as BT) strain A1470.

### BACKGROUND OF THE INVENTION

Bacillus thuringiensis was discovered as pathogenic bacteria for silkworm by a Japanese researcher, Mr. Shigetane Ishiwata, in 1901. Since then, toxic proteins produced by the bacteria have been widely utilized as bacterial insecticides for the elimination of agricultural vermin and insanitary vermin.

From the study to date, it is known that crystal proteins having an insecticidal activity are activated by solubilization under alkaline conditions, followed by protease treatment as necessary, and show toxicity to insects. It has been clarified that crystal proteins having an insecticidal activity are grouped into (1) Cry proteins having a cytocidal activity selective to insect cells upon activation by protease treatment (generally, molecular weight of the activated form is 60-75 kDa), and (2) Cyt proteins having a cytocidal activity non-selective to insect cells and a haemolytic activity to erythrocyte (generally, the molecular weight of the activated form is 22-30 kDa)

As a cell-recognizing protein, crystal proteins from Bacillus thuringiensis have excellent characteristics absent in other cell-recognizing proteins (e.g., antibody etc.), such as high selectivity represented by Cry protein, abundance of 5 diversity due to tens of genotypes, and ease in gene modification because it is a prokaryote-derived protein.

Nonetheless, its use is limited to that of an insecticide targeting insect cells. Furthermore, bacterium expressing crystal proteins having an insecticidal activity occupy only 30-40% of BT, and the nature of the crystal protein without an insecticidal activity, that are produced by BT strain, has been heretofore unclear.

Using about 2000 strains of Bacillus thuringiensis belonging to fifty kinds of serotypes H and unknown serotypes, which had been isolated from soil, cultivated soil, forest soil, sericulturist's dust, stored grain dust, insects died from illness, plants, fresh water, and the like, we found, for the first time in the world, the presence of a series of novel proteins that selectively recognize and kill vertebrate animal cells (including cancer cells) upon activation by solubilization under alkaline conditions, followed by protease treatment, in the Bacillus thuringiensis-derived toxic proteins whose activity had been unknown. This protein without an insecticidal activity, having a molecular weight of 20-70 kDa and free of a haemolytic activity to erythrocyte is described in our patent application (Japanese Patent Application No. 9-347124: entitled “Protein derived from Bacillus thuringiensis”).

### SUMMARY OF THE INVENTION

The present invention provides a series of novel proteins having a cell-recognizing activity and/or a cytopathic activity selective to vertebrate animal cells (including cancer cells) and genes encoding the proteins, which are derived from Bacillus thuringiensis, and preferably, a toxic protein produced by Bacillus thuringiensis strain A1470, and a gene encoding the protein.

Accordingly, the present invention is as follows

(1) an isolated nucleic acid molecule (for example, DNA and RNA) consisting of the nucleotide sequence represented by SEQ ID NO: 1,

(2) an isolated nucleic acid molecule encoding a protein consisting of the amino acid sequence represented by SEQ ID NO: 2,

(3) an isolated nucleic acid molecule which hybridizes under stringent conditions to a nucleic acid molecule having a nucleotide sequence complementary to the nucleotide sequence represented by SEQ ID NO: 1, and which encodes a protein having a cell-recognizing activity in an activated form,

(4) the nucleic acid molecule of (3), wherein the activated form is substantially free of a haemolytic activity,

(5) the nucleic acid molecule of (3) or (4), wherein the cell-recognizing activity is specific to at least one cell selected from the group consisting of a leukemia cell, a hepatoma cell, a uterus cancer cell and a large bowel cell,

(6) the nucleic acid molecule of any of (3)-(5), wherein the nucleic acid molecule is derived from the genus Bacillus,

(7) the nucleic acid molecule of (6), wherein the nucleic acid molecule is derived from Bacillus thuringiensis strain A1470 (deposit number: FERM P-18788)

(8) an isolated nucleic acid molecule which hybridizes under stringent condition to a nucleic acid molecule having a nucleotide sequence complementary to the nucleotide sequence represented by SEQ ID NO: 1, and which encodes a protein having a cytopathic activity in an activated form,

(9) the nucleic acid molecule of (8), wherein the activated form is substantially free of a haemolytic activity,

(10) the nucleic acid molecule of (8) or (9), wherein the cytopathic activity is specific to at least one cell selected from the group consisting of a leukemia cell, a hepatoma cell, a uterus cancer cell and a large bowel cell,

(11) the nucleic acid molecule of any of (8)-(10), wherein the nucleic acid molecule is derived from the genus Bacillus,

(12) the nucleic acid molecule of (11), wherein the nucleic acid molecule is derived from Bacillus thuringiensis strain A1470 (deposit number: FERM P-18788),

(13) a vector comprising the nucleic acid molecule of any of (1)-(12)

(14) a transformant comprising the vector of (13),

(15) an isolated protein consisting of the amino acid sequence represented by SEQ ID NO: 2,

(16) an isolated protein comprising an amino acid sequence wherein 1 or more amino acids are deleted, substituted, added or inserted in the amino acid sequence represented by SEQ ID NO: 2, whose activated form has a cell-recognizing activity,

(17) the protein of (16), whose activated form is substantially free of a haemolytic activity,

(18) the protein of (16) or (17), wherein the cell-recognizing activity is specific to at least one cell selected from the group consisting of a leukemia cell, a hepatoma cell, a uterus cancer cell and a large bowel cell,

(19) the protein of any of (16)-(18), wherein the protein is derived from the genus Bacillus,

(20) the protein of (19), wherein the protein is derived from Bacillus thuringiensis strain A1470 (deposit number: FERM P-18788),

(21) an isolated protein comprising an amino acid sequence wherein 1 or more amino acids are deleted, substituted, added or inserted in the amino acid sequence represented by SEQ ID NO: 2, whose activated form has a cytopathic activity,

(22) the protein of (21), whose activated form is substantially free of a haemolytic activity,

(23) the protein of (21) or (22), wherein the cytopathic activity is specific to at least one cell selected from the group consisting of a leukemia cell, a hepatoma cell, a uterus cancer cell and a large bowel cell,

(24) the protein of any of (21)-(23), wherein the protein is derived from the genus Bacillus,

(25) the protein of (24), wherein the protein is derived from Bacillus thuringiensis strain A1470 (deposit number: FERM P-18788),

(26) an isolated protein, which is obtained by solubilizing the protein of any of (15), (16) and (21) under alkaline conditions, followed by protease treatment,

(27) a pharmaceutical composition comprising the activated form of the protein of any of (15)-(26), or the protein of (26),

(28) a cell-recognizing agent comprising the activated form of the protein of any of (15)-(20), or the protein of (26),

(29) an anticancer agent comprising the activated form of the protein of any of (15) and (21)-(25), or the protein of (26),

(30) a method for producing the protein of any of (15)-(25), comprising the steps of:

(a) culturing a cell expressing said protein in a culture medium; and

(b) recovering said protein,

(31) an antibody directed to the protein of any of (15)-(26),

(32) a method for identifying a cell recognized by the activated form of the protein of any of (15)-(20) (or the protein of (26)), comprising the steps of:

(a) incubating the activated form of the protein with any cell in a culture medium; and

(b) determining whether the activated form of the protein shows a cell-recognizing activity to said cell,

(33) a method for identifying a cell to which the activated form of the protein of any of (15) and (21)-(25) (or the protein of (26)) shows a cytopathic activity, comprising the steps of:

(a) incubating the activated form of the protein with any cell in a culture medium; and

(b) determining whether the activated form of the protein shows a cytopathic activity to said cell,

(34) the method of (32) or (33), further comprising determining whether said protein is substantially free of a haemolytic activity,

(35) a method for identifying a substance having an affinity for the protein of any of (15)-(26) (or the protein of (26)), comprising the steps of:

(a) bringing the activated form of the protein in contact with an analyte; and

(b) determining whether said analyte has an affinity for the activated form of the protein, and the like.

### BRIEF DESCRIPTION OF THE DRAWINGS

FIG. 1 represents restriction maps of pSHAN32.1 and pSHAN32.2.

FIG. 2 represents the nucleotide sequence and the amino acid sequence of 32 kDa protein.

FIG. 3 represents SDS-PAGE and immunoblotting of 32 kDa protein before and after the protease treatment (lane S: standard protein, lane 1: BT strain A1470, lane 2: BT strain BFR1 (pHT3101), lane 3: BT strain BFR1 (pSHAN32.2)).

### DETAILED DESCRIPTION OF THE PREFERRED EMBODIMENTS

The present invention provides a protein consisting of the amino acid sequence represented by SEQ ID NO: 2. The present invention also provides proteins comprising an amino acid sequence wherein one or more amino acids are deleted, substituted, added or inserted in the amino acid sequence represented by SEQ ID NO: 2, whose activated form has a cell-recognizing activity and/or a cytopathic activity, in addition to the above-mentioned protein. Preferably, the protein of the present invention does not substantially have a haemolytic activity. Furthermore, the present invention provides a peptide consisting of at least 10, preferably at least 15, more preferably at least 20 continuous amino acid residues of the protein described above.

In the context of the present invention, the “cytopathic activity” means the ability to specifically kill a certain cell. For example, the protein of the present invention is considered to have a “cytopathic activity” to a certain cell, when the protein of the present invention shows an EC₅₀ (μg of purified protein/ml) of, for example, about 10 μg/ml or less, preferably about 5 μg/ml or less, more preferably about 1 μg/ml or less, further more preferably about 0.5 μg/ml or less, which is determined according to MTT assay as described in EXAMPLES. In addition, the protein of the present invention is considered to have a cytopathic activity to a certain cell, when the protein of the present invention shows a significantly higher cytopathic activity to the certain cell than that to a negative control, where the activity is determined according to a method other than MTT assay. Such a negative control includes any cell to which the protein of the present invention does not show a cytopathic activity. For example, any cell selected from HeLa cell, UtSMC cell, HC cell, A549 cell, MRC-5 cell or Vero cell can be used as a negative control.

Furthermore, since the protein of the present invention shows a cytopathic activity specific to a certain cell by recognizing and killing the cell, it has a specific “cell-recognizing activity”. For example, the protein of the present invention is considered to have a cell-recognizing activity to a certain cell, when the protein of the present invention shows a significantly higher cell-recognizing activity than that to a negative control, where the activity is determined according to an immunoprecipitation assay or an interaction analysis using BIAcore. Such a negative control includes any cell to which the protein of the present invention does not show a cell-recognizing activity. The cell to be used as the negative control can be appropriately selected by one of those skilled in the art.

The cell-recognizing activity can be measured by, for example, an immunoprecipitation assay, an interaction analysis using BIAcore, and the like. In the immunoprecipitation assay, a ligand (A1470 protein in the context of the present invention), an antibody directed to the ligand, and protein A bead are added to a cell lysate (containing the target of the ligand) to form a complex of the target, the ligand, the antibody and the protein A bead, and the complex is separated from the cell lysate by centrifugation. Then, the target is identified by SDS-PAGE and western blotting for the complex. A series of experimental conditions are appropriately determined by one of those skilled in the art. In the interaction analysis using BIAcore, the interaction is detected as changes in the SPR (surface plasmon resonance) signals at real time by fixing a membrane fraction containing the target present in a cell surface or the target itself on surface of a sensor, adding a specimen (1470 protein in the context of the present invention), which acts on the target, thereto via a microfluidic system, and monitoring minor changes in the mass caused by association and dissociation of molecules on the surface of the sensor. The resulting data enables kinetics-based analysis [for details of the immunoprecipitation assay, interaction analysis using BIAcore, and the like, see a separate volume of CELL TECHNOLOGY (1997), Experimental protocol series, protein experiment protocol 1 (1) functional analysis, Shujunsha]. Assays for measuring a cytopathic activity include, for example, the immunoprecipitation assay and the interaction analysis using BIAcore, but a cell-recognizing activity can be measured using other assays by one of those skilled in the art.

In the context of the present invention, “a protein, whose activated form has a cell-recognizing activity and/or a cytopathic activity” and “a protein having a cell-recognizing activity and/or a cytopathic activity in an activated form” means a protein in an inactivated form (precursor) that is converted to a form having the activity described above (i.e., activated form) when treated under certain conditions (for example, treated under alkaline conditions, or under alkaline conditions followed by protease treatment), as well as a protein having the aforementioned activity even without treatment under certain conditions (i.e., a protein originally occurred in an activated form). For example, a protein consisting of the amino acid sequence represented by SEQ ID NO: 2 generally exists in an inactivated form (32 kDa) unless subjected to a certain treatment, but once placed under alkaline conditions or placed under alkaline conditions and subjected to a treatment with protease, it is converted to an activated form (28 kDa) of the protein and exhibits a cell-recognizing activity and/or a cytopathic activity. Therefore, the protein consisting of the amino acid sequence represented by SEQ ID NO: 2 is a protein having a cell-recognizing activity and/or a cytopathic activity in its active form. A protein itself is considered to be the activated form, when the protein has a cell-recognizing activity and/or a cytopathic activity without any particular treatment.

Furthermore, the cell specifically recognized and/or killed by the protein of the present invention includes, for example, T cell leukemia cell (preferably MOLT-4 cell and Jurkat cell), myeloblast leukemia cell (preferably HL60 cell), uterus cancer cell (preferably uterus cancer cell other than HeLa cell, more preferably Sawano cell and TCS cell), hepatoma cell (preferably HepG2 cell) and large bowel cancer cell (preferably colon cancer cell, more preferably Caco-2 cell), which are derived from human, and mouse fibroblast (preferably NIH3T3 cell). Nonetheless, one of those skilled in the art will understand that the protein of the present invention has a cell-recognizing activity and/or a cytopathic activity to a cell other than the cells described above. Furthermore, the protein of the present invention may have one of these activities alone or in a combination of these. For example, the protein of the present invention has a cytopathic activity to T cell leukemia cell and large bowel cancer cell, or alternatively, a cytopathic activity to T cell leukemia cell and a cell-recognizing activity to large bowel cancer cell. The protein of the present invention includes an inactivated form (32 kDa protein) consisting of the amino acid sequence represented by SEQ ID NO: 2, as well as a protein obtained by treating said protein under alkaline conditions and an activated form (28 kDa protein) obtained by treating said protein under alkaline conditions, followed by a protease treatment. The conditions and procedures of the alkaline treatment and the protease treatment can be determined by one of those skilled in the art, and whether an activated form (28 kDa protein) could be obtained can be confirmed by SDS-PAGE and the like. Specifically, the conditions and the procedures of the treatment are as described in EXAMPLES.

The “substantially free of haemolytic activity” means that the protein of the present invention does not substantially show a haemolytic activity to mammalian erythrocyte (for example, rabbit, sheep, human and the like, preferably human). Specifically, the protein of the present invention is considered to be substantially free of a haemolytic activity, when the difference between the absorbance of the protein of the present invention and the absorbance in a control experiment is 0.5 or less, where the absorbance is determined according to the method described in EXAMPLE 6. As the protein of the present invention, a protein having a cell-recognizing activity and/or a cytopathic activity but substantially free of a haemolytic activity is preferred. Preferably, the protein of the present invention is preferably a protein derived from the genus Bacillus, more preferably a protein derived from Bacillus thuringiensis, further preferably a protein derived from Bacillus thuringiensis strain A1470 (deposit number: FERM P-18788, deposited at the International Patent Organism Depository, National Institute of Advanced Industrial Science and Technology (Tsukuba Central 6, 1-1 Higashi 1-chome Tsukuba-shi, Ibaraki-ken Japan), deposited date: Mar. 20, 2002).

Other cell-recognizing proteins other than the protein of the present invention include, for example, an immunoglobulin. The protein of the present invention is advantageous over immunoglobulin in that it permits easy modification, because it has a considerably lower molecular weight as compared to immunoglobulin and is derived from prokaryote. The protein of the present invention can be utilized for a drug delivery system in view of the characteristics described above and the high selectivity it has. For example, the protein of the present invention can be fused with other substance to confer specific directivity on the substance. A person will understand that the protein of the present invention can be used in targeting therapy as similar to the immunoglobulin (for example, antibody), since it has a superior selectivity.

The protein of the present invention can be used as an anticancer agent. The protein of the present invention is effective by itself for use as an anticancer agent.

Furthermore, the protein of the present invention can be used as a conjugate (covalently or non-covalently bonded) with, or in combination with, other anticancer drug for improving the effectiveness. For example, the conjugate of an immunoglobulin and an anticancer drug has been reported. Similarly, the protein of the present invention can be used as a conjugate with other anticancer drug. In addition, the protein of the present invention can be labeled with a radioisotope (for example, ¹³¹I). For example, a report has documented that a therapy using an anti-CD20 monoclonal antibody labeled with ¹³¹I showed extremely fine results in patients suffering from B cell lymphoma. Therefore, the protein of the present invention, too, could have a potentiated killing effect on cancer cells upon labeling with ¹³¹I. Furthermore, the protein of the present invention can be used as a conjugate with toxin (for example, ricin) For example, there is a report on a confirmed marked effect in patients suffering from T cell lymphoma by a conjugate of an anti-CD5 antibody and ricin A chain. Therefore, the protein of the present invention can be also used as a conjugate with a ricin A chain.

The protein of the present invention can be used as a cell-recognizing agent. In in vivo application, for example, tumor (consisting of certain cancer cells) can be imaged by labeling the protein of the invention with a suitable imaging substance (for example, a substance that can be detected by nuclear magnetic resonance). The radioisotope used in the imaging of tumor includes, for example, yttrium, indium, technetium and the like. A radiation dosage administered to the subject can be appropriately determined by one of those skilled in the art. In in vitro application, for example, the protein of the present invention can be used as a marker for a certain cell.

The present invention provides a nucleic acid molecule encoding a protein consisting of the amino acid sequence represented by SEQ ID NO: 2, preferably a nucleic acid molecule consisting of the nucleotide sequence represented by SEQ ID NO: 1. The present invention also provides a nucleic acid molecule which hybridizes under stringent conditions to a nucleic acid molecule having a nucleotide sequence complementary to the nucleotide sequence represented by SEQ ID NO: 1, and which has a cell-recognizing activity and/or a cytopathic activity, and preferably being substantially free of a haemolytic activity.

Furthermore, the present invention provides an oligonucleotide consisting of at least about 15, preferably at least about 16, more preferably at least about 18, most preferably at least about 20, continuous nucleotides of the nucleic acid described above. The present invention also provides an antisense oligonucleotide complementary to the oligonucleotide. The present invention further provides a pair of primers for amplifying the nucleic acid of the present invention. The pair of primers is two oligonucleotides, each of which having at least 15, preferably at least 20, continuous oligonucleotides, and the two primers being complementary to sense strand and antisense strand, respectively.

In the context of the present invention, the “stringent conditions” means the conditions under which a nucleic acid having about 60% or more, preferably about 70% or more, more preferably about 75% or more, further preferably about 80% or more, further more preferably about 85% or more, about 95% or more, about 95% or more, about 96% or more, about 97% or more or about 98% or more, most preferably about 99% or more, identity can be hybridized. The stringency can be controlled by appropriately varying salt concentrations and temperatures of the hybridization reaction and washing. One example of stringent hybridization conditions are hybridization in 6×sodium chloride/sodium citrate (SSC) at about 45° C., followed by one or more low stringency washes in 0.2×SSC/0.1% SDS at room temperature, or by one or more moderate stringency washes in 0.2×SSC/0.1% SDS at 42° C., or washed in 0.2×SSC/0.1% SDS at 65° C. for high stringency.

The degree of the identity can be calculated by using, for example, BLAST search of National Center for Biotechnology Information (NCBI) with a default setting. In yet another embodiment, the degree of the identity between two nucleotide sequences is determined using the GAP program in the GCG software package (Devereux et al. (1984) Nucleic Acids Res. 12(1):387) (available at www.gcg.com), using a NWSgapdna.CMP matrix and a gap weight of 40, 50, 60, 70, or 80 and a length weight of 1, 2, 3, 4, 5, or 6.

A cell, to which the protein of the present invention shows a cytopathic activity, can be identified by a method comprising (a) incubating the protein of the present invention with any cell in a culture medium, and (b) determining whether said protein shows a cytopathic activity to the cell. Preferably, the identification method can optionally comprise determining whether said protein is substantially free of a haemolytic activity. A cell is considered to be a cell, to which the protein of the present invention shows a cytopathic activity, when the protein of the present invention shows EC₅₀ (μg of purified protein/ml) of, for example, about 10 μg/ml or less, preferably about 5 μg/ml or less, more preferably about 1 μg/ml or less, further more preferably about 0.5 μg/ml or less, to the cell, which is determined according to MTT assay as described in EXAMPLES. In addition, a cell is considered to be a cell, to which the protein of the present invention shows a cytopathic activity, when the cell is more significantly killed than in a negative control by the protein of the present invention, which is determined according to a method other than the MTT assay. Such negative control includes any cell to which the protein of the present invention does not show a cytopathic activity. For example, any cell selected from the group consisting of-HeLa cell, UtSMC cell, HPC cell, A549 cell, MRC-5 cell and Vero cell can be used-as a negative control. A positive control includes any cell to which the protein of the present invention shows a cytopathic activity. Preferably, any cell selected from the group consisting of MOLT-4 cell, Jurkat cell, HL60 cell, Sawano cell, TCS cell, HepG2 cell, Caco-2 cell and NIH3T3 cell can be used as a positive control. A cell, which is killed to the same level as or more than the cell of the positive control, is more preferable as a cell to which the protein of the present invention shows the cytopathic activity. For example, the protein of the present invention can be applied for treating diseases relating to the cell identified by this method.

A cell, to which the protein of the present invention shows a cell-recognizing activity, can be identified by a method comprising (a) incubating said protein with any cell in a culture medium, and (b) determining whether said protein has a cell-recognizing activity to the cell. Preferably, the identification method can optionally comprise determining whether said protein is substantially free of a haemolytic activity. For example, a cell is considered to be a cell to which the protein of the present invention shows a cell-recognizing activity, when the cell is more significantly recognized than the cell of a negative control by the protein of the present-invention, which is determined according to an immunoprecipitation assay or an interaction analysis using BIAcore. A negative control includes any cell to which the protein of the present invention does not show a cell-recognizing activity. A cell to be used as a negative control can be appropriately selected by one of those skilled in the art. A positive control includes any cell to which the protein of the present invention shows a cell-recognizing activity. Preferably, any cell selected from MOLT-4 cell, Jurkat cell, HL60 cell, Sawano cell, TCS cell, HepG2 cell, Caco-2 cell or NIH3T3 cell can be used as a positive control. A cell, which is recognized to the same level as or more than the cell of the positive control, is more preferable as a cell to which the protein of the present invention shows the cell-recognizing activity. For example, the protein of the present invention can be applied for treating diseases relating to the cell identified by this method.

The present invention provides a method for identifying a substance having an affinity for the protein of the present invention. The method comprises (a) bringing said protein in contact with an analyte, and (b) determining whether said analyte has affinity for said protein. Such analyte includes, but not limited to, saccharides, lipids, proteins, nucleic acids, synthetic compounds and the like. Preferable affinity is that showing a dissociation constant (Kd) of 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less. Such affinity can be measured by, for example, labeling the protein of the present invention with a radioisotope, and subjecting the labeled protein to a binding assay with an analyte. For example, a substance having an affinity for the protein of the present invention can be utilized for purification of the protein of the present invention.

The present invention also provides an antibody having a specific affinity against the protein of the present invention. The antibody may be a polyclonal antibody or monoclonal antibody, and can be prepared according to an immunological method well known in the art. The antibody may be not only a complete antibody molecule, but also any fragment as long as it has an antigen-binding site (CDR) against the protein of the present invention. Such fragment includes, for example, Fab, F(ab′)₂, ScFv, minibody, and the like. For example, the antibody of the present invention can be labeled with horseradish peroxidase etc and used for detecting whether the protein of the present invention binds to a certain cell.

For example, a polyclonal antibody can be obtained by administering the protein of the present invention or a fragment thereof (where necessary, a conjugate bridged to a carrier protein such as bovine serum albumin, KLH (Keyhole Limpet Hemocyanin) and the like) as an antigen to an animal subcutaneously or intraperitoneally along with a commercially available adjuvant (for example, Freund's complete or incomplete adjuvant) 2 to 4 times every 2 or 3 weeks (antibody titer of serum partially collected from the blood should have been measured by a known antigen-antibody reaction to confirm increase thereof), sampling the whole blood about 3 to 10 days after the final immunization, and purifying anti-serum from the blood. The animal to be subjected to administration of the antigen includes mammals such as rat, mouse, rabbit, goat, guinea pig, hamster and the like.

A monoclonal antibody can be generated by a cell-fusion method. For example, the antigen is administered to a mouse subcutaneously or intraperitoneally along with a commercially available adjuvant 2 to 4 times, and the spleen or lymph node is taken 3 days after the final administration, from which leukocyte is harvested. The leukocyte and a myeloma (for example, NS-1, P3X63Ag8 etc.) are fused to obtain a hybridoma producing monoclonal antibody against the antigen. The cell fusion method may be PEG method or pulsed electric field method. A hybridoma producing desired monoclonal antibody can be selected by detecting an antibody which binds with an antigen in culture supernatant by EIA, RIA or the like well known in the art. A hybridoma producing the monoclonal antibody can be cultured in vitro, or in vivo, such as in ascites of mouse or rat, preferably mouse. The antibody can be obtained from culture supernatant or ascites of animal.

The present invention also comprises a method for processing information of the nucleic acid sequence and the amino acid sequence of the present invention on computer. For example, the present invention provides a search for nucleic acid sequences and/or amino acid sequences having homology with the nucleotide sequence and/or the amino acid sequence of the present invention by using the nucleic acid sequence and the amino acid sequence of the present invention (for example, use of BLAST and FASTA program). The present invention also provides obtainment of more optimal sequence information by using information of the nucleic acid sequence and the amino acid sequence of the present invention. The present invention further provides a nucleic acid and a protein consisting of certain sequence obtained using the information of the nucleic acid sequence and the amino acid sequence of the present invention.

The present invention also provides a recombinant vector containing a DNA encoding the protein of the present invention. The recombinant vector of the present invention is not particularly limited as long as it can be maintained by replication or autonomously replicated within various host cells, such as prokaryotic cells and/or eukaryotic cells, and includes plasmid vectors, viral vectors and the like. The recombinant vector can be easily prepared by ligating the above-mentioned DNA encoding the protein of the present invention to a known cloning vector or expression vector available in the art, using suitable restriction enzymes and a ligase, and further, linkers and adaptors as necessary.

Examples of such vector include pBR322, pBR325, pUC18, pUC19 etc. as a plasmid derived from Escherichia coli; pSH19, pSH15 etc. as a plasmid derived from yeast; and pUB110, pTP5, pC194 etc. as a plasmid derived from Bacillus subtilis. Examples of the plasmid derived from Bacillus thuringiensis include pHT3101, pHT315 and the like. Examples of the virus include bacteriophages such as λ phage, and animal and insect viruses such as parvovirus (SV40, bovine papilloma virus (BPV) etc.), retrovirus (Moloney murine leukemia virus (MoMuLV) etc.), adenovirus (AdV), adeno-associated virus (AAV), vacciniavirus, vaculovirus, and the like.

Particularly, the present invention provides an expression vector in which a DNA encoding the protein of the present invention is placed under the control of a promoter functional in a desired host cell. The vector to be used is not particularly limited as long as it contains a promoter region, which is capable of functioning in various host cells such as prokaryotic and/or eukaryotic cells and regulating the transcription of a gene located-at its downstream (e.g., when the host is Escherichia coli, trp promoter, lac promoter, lecA promoter, etc., when the host is Bacillus subtilis, SPO1 promoter, SPO2 promoter, penP promoter, etc., when the host is yeast, PHO5 promoter, PGK promoter, GAP promoter, ADH promoter, etc., and when the host is a mammalian cell, viral promoters such as SV40 early promoter, MoMuLV long terminal repeat, adenovirus early promoter, etc.), and a termination signal of the transcription of said gene, i.e., terminator region, wherein the promoter region and the terminator region are ligated via a sequence containing at least one restriction enzyme recognition site, preferably a unique restriction site that cleaves the vector only at this site. However, it is preferable that it further contain a selectable marker gene for selecting transformants (e.g., a gene imparting resistance to a drug such as tetracycline, ampicillin, kanamycin, hygromycin and phosphinothricin, a gene complementing auxotrophic mutation etc.). Moreover, when the DNA encoding the protein of the present invention does not contain an initiation codon or a termination codon, a vector which contains an initiation codon (ATG or GTG) and a termination codon (TAG, TGA or TAA) at the downstream of the promoter and the upstream of the terminator, respectively, is preferably used.

When bacteria is used as a host cell, in general, the lo expression vector needs to contain a replicable unit which allows autonomous replication in the host cell, in addition to the above-mentioned promoter region and terminator region. The promoter region also contains an operator and Shine-Dalgarno (SD) sequence near the promoter.

When the protein of the present invention is secreted into a culture medium of the transformant but DNA encoding the protein does not have a sequence encoding signal peptide, a secretory expression vector, further containing a suitable signal codon following an initiation codon, is preferably used as a vector.

When the DNA encoding the protein of the present invention is isolated from a genomic DNA and obtained together with its native promoter and terminator regions, the expression vector of the present invention can be prepared by inserting the DNA into a suitable site of a known cloning vector which can be maintained by replication or which can be autonomously replicated in a desired host cell.

The present invention also provides a transformant obtained by transforming a host cell with the above-mentioned expression vector. The host cell to be used in the invention is not particularly limited as long as it is capable of adapting to the above-mentioned expression vector and can be transformed therewith, and is exemplified by various cells such as naturally occurring cells or artificially established mutant or recombinant cells conventionally used in the technical field of the present invention [e.g., bacteria (Escherichia coli, Bacillus subtilis, lactobacillus, Bacillus thuringiensis or mutant thereof (preferably, Bacillus thuringiensis strain which does not produce toxic protein)etc.), yeast (Saccharomyces, Pichia, Kluyveromyces etc.), animal cell and insect cell]. Preferably, prokaryote can be used. Bacillus thuringiensis mutant expressing the protein of the present invention appropriately can be generated by one of those skilled in the art. For example, Bacillus thuringiensis strain which does not produce a toxic protein can be obtained by curing Bacillus thuringiensis strain producing a toxic protein and removing plasmid DNA from the strain. Any Bacillus thuringiensis strain, which does not produce a toxic protein and produce the protein of the present invention can be obtained according to the method described above. (see, Gonzalez, J. M. et al. PLASMID 5, 351-365 (1981)).

The expression vector can be introduced into a host cell using a method known in the art. For example, the method of Cohen et al. [Proc. Natl. Acad. Sci. USA., 69, 2110 (1972)], protoplast method [Mol. Gen. Genet., 168, 111 (1979)] and competent method [J. Mol. Biol., 56, 209 (1971)] can be used for bacteria; the method of Hinnen et al. [Proc. Natl. Acad. Sci. USA., 75, 1927 (1978)] or Lithium method [J. Bacteriol., 153, 163 (1983)] can be used for yeast; the method of Graham [Virology, 52, 456 (1973)] can be used for animal cell; and the method of Summers et al. [Mol. Cell. Biol., 3, 2156-2165 (1983)] can be used for insect cell, for transformation.

The protein of the present invention can be obtained from cell expressing the protein of the present invention. Cell expressing the protein of the present invention include, for example, cell naturally expressing the protein of the present invention (preferably Bacillus thuringiensis strain A1470), and transformant comprising the expression vector encoding the protein of the present invention. The protein of the present invention can be produced by culturing the transformant comprising the expression vector prepared as described above, and recovering the protein of the present invention from the resulting culture. The protein of the present invention also can be obtained by culturing the cell naturally expressing the protein of the present invention (preferably Bacillus thuringiensis strain A1470), and recovering the protein of the present invention. Preferably, the protein of the present invention is activated after solubilization with alkaline buffer, optionally followed by protease treatment.

The medium to be used preferably contains carbon source and inorganic or organic nitrogen source necessary for the growth of host cell (transformant). Examples of the carbon source include glucose, dextran, soluble starch and sucrose; -examples of the inorganic or organic nitrogen source include ammonium salts, nitrates, amino acids, corn steep liquor, peptone, casein, meat extract, soybean lees, potato extract solution and the like. Where desired, other nutrient sources such as inorganic salts (e.g., calcium chloride, sodium dihydrogenphosphate and magnesium chloride), vitamins and antibiotics (e.g., tetracycline, neomycin, ampicillin and kanamycin) may be added.

Culture is performed by a method known in the art. Specific examples of the medium and culture conditions to be used depending on the host cell are shown in the following, which should not be construed as limiting the medium and culture conditions of the invention.

When the host is bacteria, actinomyces, yeast or fungus, a liquid medium containing the aforesaid nutrient sources is suitable, with preference given to a medium having a pH of 5 to 8. When the host is Escherichia coli, preferable medium include LB medium and M9 medium [Miller. J., Exp. Mol. Genet, p.431, Cold Spring Harbor Laboratory, New York (1972)]. In this case, culture can be typically performed at 14° C. to 43° C. for about 3 to 24 hr with aeration and agitation as necessary.

When the host is Bacillus subtilis, culture can be typically performed at 30° C. to 40° C. for about 16 to 96 hr with aeration and agitation as necessary. When the host is Bacillus thuringiensis or mutant thereof (preferably Bacillus thuringiensis BFR1), preferable medium include CYS medium (Yamamoto, T., ACS Symp. Ser. 432, 46-60 (1990))(in this case, for example, culture can be performed on agar plate at 20° C. for 96hr.). When the host is yeast, examples of the medium include Burkholder minimum medium [Bostian. K. L. et al., Proc. Natl. Acad. Sci. USA, 77, 4505 (1980)], and pH is preferably 5 to 8. Culture can be typically performed at 20° C. to 35° C. for about 14 to 144 hr with aeration and agitation as necessary.

When the host is animal cells, examples of the medium include minimum essential medium (MEM) containing about 5 to 20% fetal calf serum [Science, 122, 501 (1952)], Dulbecco's modified minimum essential medium (DMEM) [Virology, 8, 396 (1959)], RPMT1640 medium [J. Am. Med. Assoc., 199, 519 (1967)], 199 medium [Proc. Soc. Exp. Biol. Med., 73, 1 (1950)] and the like. The pH of the medium is preferably about 6 to 8. Culture is typically performed at 30° C. to 40° C. for about 15 to 72 hr with aeration and agitation as necessary.

The protein of the present invention can be purified by an appropriately combining various separation techniques conventionally used. The protein of the present invention can be obtained by appropriately selecting known separation methods such as salting out, solvent precipitation, dialysis, ultrafiltration, gel filtration, non-denatured polyacrylamide gel electrophoresis (PAGE), sodium dodecyl sulfate-polyacrylamide gel electrophoresis (SDS-PAGE), ion exchange chromatography, hydroxyapatite chromatography, affinity chromatography, reversed phase high performance liquid chromatography, isoelecric focusing and the like. As a means for obtaining the recombinant protein of the present invention rapidly and easily, preferably exemplified is a method which comprises adding a DNA sequence encoding an amino acid sequence capable of adsorbing to a metal ion chelate (e.g., a sequence consisting of basic amino acids such as histidine, arginine or lysine, preferably histidine) to a certain region (preferably C terminus) of coding sequence for the protein of the present invention by genetic manipulation, allowing expression within a host cell, and recovering the protein from an active fraction of the protein in the cell culture by separation utilizing its affinity for a carrier immobilizing said metal ion chelate. The DNA sequence encoding an amino acid sequence capable of adsorbing to a metal ion chelate can be introduced into the coding sequence by, for example, performing PCR amplification using a hybrid primer comprising said DNA sequence linked to the nucleotide sequence encoding the C-terminal amino acid sequence of the protein of the present invention, in the process of cloning DNA encoding the protein, or by inserting the DNA encoding the protein in frame into an expression vector containing said DNA sequence before the termination codon. The metal ion chelate adsorbent to be used for purification is prepared by bringing a solution containing a transition metal (e.g., divalent ion of cobalt, copper, nickel or iron, or trivalent ion of iron or aluminum, preferably divalent ion of cobalt or nickel) into contact with a ligand (e.g., a matrix onto which iminodiacetate (IDA) group, nitrilotriacetate (NTA) group or tris(carboxymethyl) ethylenediamine (TED) group is attached) to allow binding thereof with the ligand. The matrix part of the chelate adsorbent is not particularly limited as long as it is a conventional insoluble carrier.

The present invention provides pharmaceutical compositions, specifically anticancer agent and cell-recognizing agent, comprising the protein, expression vector or transformant as active ingredient. The present invention also provides an agent for treating immune disorder, preferably an agent for treating disease due to cancerous (abnormal) T cell, more preferably an agent for treating leukemia, further lo preferably an agent for treating leukemia due to abnormal T cell. Immune cell (preferably T cell) to which the protein of the present invention shows a cell-recognizing activity and/or a cytopathic activity can be determined according to the identification method described above by one of those skilled in the art.

In the context of the present invention, by the “composition” and “agent” is meant one containing a specific substance as an active ingredient. For example, the pharmaceutical composition of the present invention comprises the protein of the present invention as an active ingredient, thereby showing a therapeutic effect on certain diseases. Such pharmaceutical composition can be used as a pharmaceutical preparation in the form of a solid, a semi-solid or a solution, containing the protein of the present invention as an active ingredient admixed with an organic or inorganic carrier, or an excipient suitable for oral or parenteral administration. The active ingredient can be mixed with a normal, atoxic, pharmaceutically acceptable carrier for powder, tablet, pellet, capsule, suppository, solution, emulsion, suspension, aerosol, spray or other form suitable for use. Furthermore, adjuvant, stabilizer, thickener or the like can be optionally used. Such carrier and excipient may be aseptically treated before use, if necessary. In addition, a pharmaceutical composition containing such carrier and excipient can be aseptically treated. While the therapeutically effective amount of the protein of the present invention as an active ingredient depends on its purity, activity and the mode of administration, as well as the age, health, body weight, sexuality and the kind of the disease of each patient to be treated, the dose can be appropriately determined by one of those skilled in the art in, view of these factors.

Before the proteins, nucleic acids, methods and the like of the present invention are disclosed and described, it is to be understood that the present invention is not limited to the specific embodiment described herein. It is to be further understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

### EXAMPLES

The present invention is further explained in detail by way of Examples in the following. These are mere examples, which in no way limit the scope of the present invention.

Materials

Cells used in this experiments were purchased as follows. HeLa cell (human cervical cancer cell), Sawano cell (human endometrium adenocarcinoma cell), TCS cell (human keratinizing type squamous carcinoma cell), HepG2 cell (hepatoma cell), MOLT-4 cell (human T cell leukemia cell), Jurkat cell (human T cell leukemia cell), HL60 cell (human myeloblast leukemia cell), MRC-5 cell (human embryonic lung fibroglast), A549 cell (human lung cancer cell), Caco-2 cell (human colon cancer cell), COS-7 cell (Africa green monkey kidney cell), Vero cell (Africa green moneky kidney cell) and NIH3T3 cell (mouse embryonic fibroblast) were purchased from the Institute of Physical and Chemical Research. UtSMC cell (normal human uterus smooth muscle cell) was purchased from Takara Shuzo Co., Ltd. HC cell (normal human hepatic cell) was purchased from CellSystems. Sheep blood was purchased from the Nippon Bio-Test Laboratory Inc.

### Example 1

Cloning of the Gene

A 32 kDa protein derived from Bacillus thuringiensis strain A1470 (deposit number: FERM P-18788) (hereinafter abbreviated as BT strain A1470) was transfered to PVDF membrane (Bio-Rad, Hercules, Calif., U.S.A.), and the N-terminal sequence of the protein was determined using protein sequencer Model 473A (Applied Biosystems, Foster, Calif., U.S.A.). The N-terminal amino acid sequence consisting of 18 amino acid residues (AIINLLRELEIYGMQYAN) (SEQ ID NO: 3) was used to design 20-mer degenerate oligonucleotide probe, GAAATT (A) TATGGT (A) ATGCAATA (32 kDa F2 oligonucleotide) (SEQ ID NO: 4). The sequence of this oligonucleotide corresponds to E (10th)−Y (16th) of the N-terminal sequence of the 32 kDa protein.

Then, an amino acid sequence other than the N-terminal sequence was determined according to the method of Cleveland et al. (1997). The protein was treated with Staphylococcus aureus V8 protease (Roche Diagnostics, Mannheim, Germany), and the resulting internal peptides were separated on 15% SDS-PAGE. The N-terminal sequence of one of the internal peptides separated was determined according to the method described above. The amino acid sequence consisting of initial 19 amino acid residues (GAVTNENKYTLDATQDFRD) (SEQ ID NO: 5) was used to design 20-mer degenerate oligonucleotide probe, GTATATTTATTTTCATTT(A) GT (32 kDa R2 oligonucleotide) (SEQ ID NO: 6) This oligonucleitode sequence corresponds to T (10th)−T (4th) of the amino acid sequence consisting of the 19 amino acid residues described above.

Then, PCR was carried out for all plasmids from BT strain A1470 using the 32 kDa F2 oligonucleitide and the 32 kDa R2 oligonuclotide. As a result, a PCR product of 400 bp was obtained and the product was purified. The 400 bp PCR product purified was then labeled with a peroxidase by ECL nucleic acid labeling system (Amercham Pharmacia Biotech, Uppsala, Sweden) in order to generate a labeled probe. All plasmid DNAs contained in BT strain A1470 were extracted, and the plasmid DNAs were digested into fragments by restriction enzyme (ClaI) treatment. Of the fragments, about a 6.6 kb ClaI fragment was specifically hybridized with the probe labeled with the peroxidase.

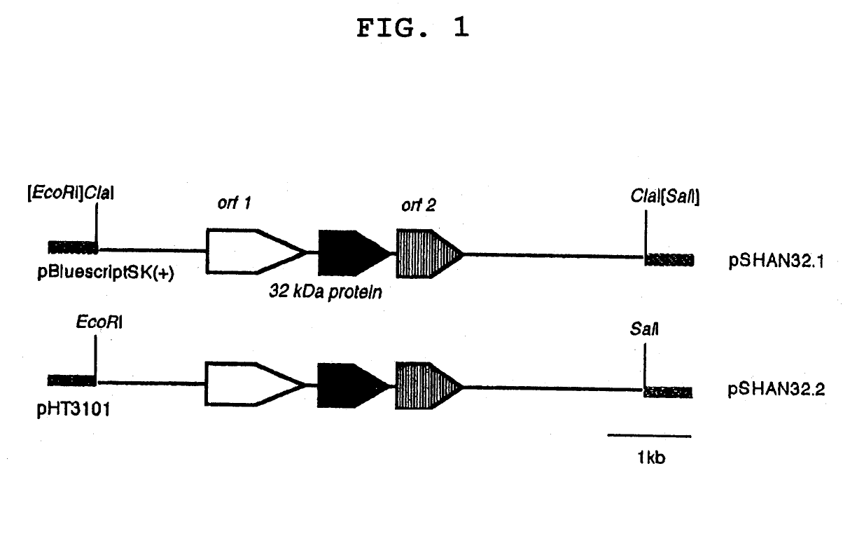

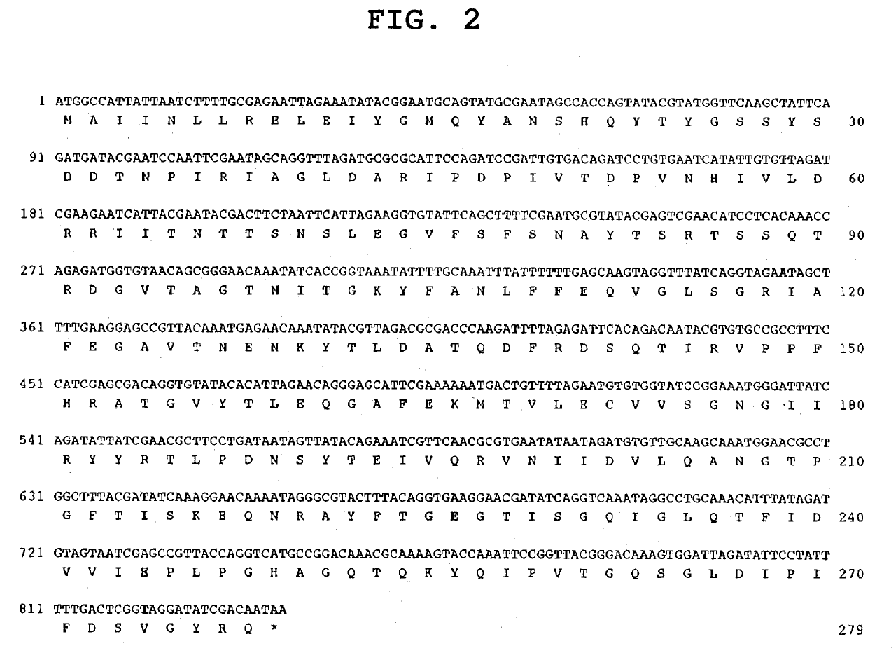

Fragments (5-7 kb) from BT strain A1470 plasmid were inserted into ClaI site of pBluescript SK(+). Escherichia coli DH5α was transformed with the inserted plasmid, and the E. coli DH5α transformed was confirmed to have hybridized with the labeled probe. The E. coli DH5α contained pSHAN32.1 plasmid. The restriction map of this plasmid is shown in FIG. 1. This plasmid contained a 6.6 kb DNA fragment comprising orf1, orf2 and a gene of the 32 kDa protein. The nucleotide sequence (SEQ ID NO: 1) and the deduced amino acid sequence (SEQ ID NO: 2) of this 32 kDa protein are shown in FIG. 2.

The EcoRI-SalI site of the pSHAN32.1 was digested to prepare a 6.6 kb fragment. This 6.6 kb EcoRI-SalI fragment was ligated to EcoRI-SalI site of E. Coli-Bacillus thuringiensis shuttle vector pHT3101 (Lereclus et al., 1989) to generate a plasmid pSHAN32.2. The plasmid pSHAN32.2 was then introduced into Bacillus thuringiensis strain BFR1 (hereinafter abbreviated as BT strain BFR1) not producing Cry protein, thereby generating a recombinant strain (BT strain BFR1 (pSHAN32.2)). Then, the 32 kDa protein encoded by the fragment described above was expressed.

### Example 2

Preparation of Activated Protein

The protein was prepared as follows. First, 40 μl of a BT strain BFR1 (pSHAN32.2) solution (containing 320 μg of the crystal protein) was washed three times with 1 ml of distilled water. An alkaline buffer (240 μl) composed of 50 mM Na₂CO₃ (pH 10.8), 10 mM DTT and 1 mM EDTA was added thereto, and the protein was solubilized at 37° C. for 1 hr. Then, supernatant containing the protein was recovered by centrifugation, and optionally, the protein was further activated by the addition of proteinase K to a final concentration of 30 μg/ml. Thereafter, PMSF was added to the protein solution to a final concentration of 1 mM. The pH of the protein solution was re-neutralized with 1N HCl and bacteria was removed by filtration to give a solution of activated protein. For preservation, the protein was frozen by the addition of glycerol.

### Example 3

SDS-PAGE and Western Blotting

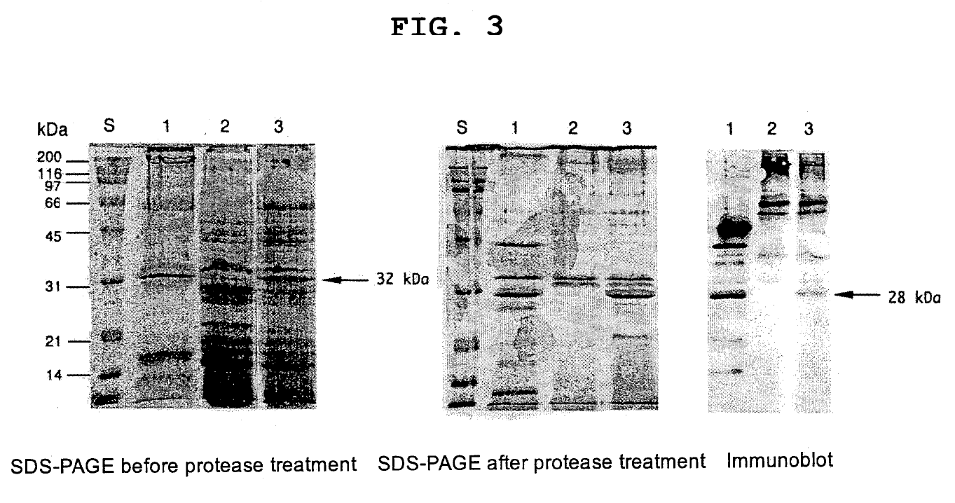

For the purpose of examining the protein expressed from BT strain BFR1 (pSHAN32.2), profiles of the proteins produced by BT strain A1470, BT strain BFR1 (pHT3101) and BT strain BFR1 (pSHAN32.2) were compared between (a) before proteinase K treatment and (b) after proteinase K treatment. Electrophoresis was carried out using 15% running gel and 4% stacking gel, and a molecular marker from Bio-Rad (200, 116, 97, 66, 45, 31, 21, 14 and 6 kDa) was used. Furthermore, western blotting was carried out for protein after protease treatment. Proteins after electrophoresis were transferred to PVDF membrane (Bio-Rad, Hercules, Calif., U.S.A.), and primary antibody (antibody against the activated form of the 28 kDa protein) and secondary antibody (peroxidase-labeled anti-rabbit IgG) were used to detect the activated protein. The results are shown in FIG. 3. The results shown in FIG. 3 indicate that BT strain BFR1 (pSHAN32.2) (recombinant strain) produced a protein having the same molecular weight (32 kDa) as that of BT strain A1470 (wild type strain). It has been also indicated that the 28 kDa protein (activated form) occurred by solubilizing the 32 kDa protein with alkaline buffer, followed by proteinase K treatment, as similar to BT strain A1470 (wild type strain).

### Example 4

Preparation of Cell Culture

MOLT-4 cells were cultured in RPMI1640 medium at 37° C., 5% CO₂, according to a conventional method, and poured into a 96-well multi-well plate by 90 μl at a concentration of 2.2×10⁵ cells/ml. MOLT-4 cells were further cultured in the 96-well multi-well plate, and used in the assay.

HeLa cells were cultured in MEM medium at 37° C., 5% Co₂, according to a conventional method, and poured into a 96-well multi-well plate by 90 μl at a concentration of 2.2×10⁵ cells/ml. HeLa cells were further cultured in the 96-well multi-well plate, and used in the assay.

### Example 5

Measurement of Cytopathic Effect (CPE) on Cancer Cell and Cell Viability

The cytopathic activities of 28 kDa proteins derived from wild type strain (BT strain A1470) and recombinant strain (BT strain BFR1 (pSHAN32.2)) were measured according to CPE (change of cell morphology) and cell viability based on MTT assay. A 32 kDa protein was obtained according to the method of EXAMPLE 1. The 32 kDa protein was activated by solubilization with an alkaline buffer, followed by a proteinase K treatment. Then, the resulting 28 kDa protein (activated form) was purified with an ion-exchange resin. The purified 28 kDa protein was added to the cell culture prepared in EXAMPLE 4 in each well of the 96-well multi-titer plate to a final concentration of 1 μg/ml. Observation on CPE and measurement of cell viability were carried out 20 hr after the addition of the protein. CPE was observed using an inverted microscope, and estimated objectively based on the proportion of the cells subject to cytopathy in 1000 cells (5% or less: −, 5−10%: ±, 10−30%: +, 30−60%: ++, 60% or more: +++) (Mizuki,E. et al. J. appl. Microbiol. 86, 477-486 (1999)). The cell viability was measured according to MTT assay using commercially available CellTiter 96™ reagent. The results are shown in Table 1.

TABLE 1
Cytocidal effect of the protein (28 kDa) purified
from BT strain on human cell lines

| | | BT strain | | | |
|---|---|---|---|---|---|
| | | A1470 | | BFR1 (pSHAN32.2) | |
| cell line | | (wild-type) | | (recombinant) | |
| (tissue) | cell type | CPE | viability (%) | CPE | viability (%) |
| MOLT-4) | T cell leukemia | +++ | 10 | ++ | 31 |
| (blood) | | | | | |
| HeLa | cervical cancer | − | 100 | − | 100 |
| (cervical) | | | | | |

The results of both the CPE and MTT assays shown in Table 1 indicate that the 28 kDa protein derived from BT strain BFR1 (pSHAN32.2) (recombinant strain) did not show a cytopathic activity to HeLa cell but showed a cytopathic activity to MOLT-4 cell, as similar to the 28 kDa protein derived from BT strain A1470 (wild type strain)

### Example 6

Measurement of Haemolytic Activity

Sheep blood pre-prepared was diluted with 20 mM Tris buffer to an erythrocyte concentration of 2% (v/v). The activated protein solution prepared in EXAMPLE 2 was added to the 40 μl of the 2% (v/v) solution and the mixture was stood at 27° C. for 18 hr. Then, the solution was centrifuged at 800×g for 10 min, and absorbance (540 nm) of the supernatant was measured. In control experiment, 20 mM Tris buffer was added instead of the protein solution. The protein solution was considered to be free of a haemolytic activity, when the difference between the absorbance of the protein solution and the absorbance in control experiment was 0.5 or less. The results are shown in Table 2.

TABLE 2
Haemolytic activity of the protein (28 kDa) purified
from BT strain A1470

| | haemolytic activity |
|---|---|
| Orgin of protein | (sheep erythrocyte) |
| A1470 | — |
| BFR1 strain (pSHAN32.2) | — |

The results shown in Table 2 indicate that the 28 kDa protein derived from BT strain BFR1 (pSHAN32.2) (recombinant strain) was free of a haemolytic activity to the sheep blood, as similar to the 28 kDa protein derived from BT strain A1470 (wild-type strain).

### Example 7

Effect of Protein of the Present Invention on Various Cell Lines

Various cell lines were cultured according to the conditions shown in Table 3, and the effects of the 28 kDa protein of the present invention on each cultured cell line were evaluated. The culture conditions for each cell line are shown in Table 3, and the results of cytopathic activity to each cell line are shown in Table 4.

TABLE 3
Culture conditions for each cell line

| cell line | medium/additive | temprature | CO₂ conc. |
|---|---|---|---|
| MOLT-4 | RPMI1640/10% FBS | 37° C. | 5% |
| Jurkat | RPMI1640/10% FBS | 37° C. | 5% |
| HL-60 | RPMI1640/10% FBS | 37° C. | 5% |
| HeLa | MEM/10% CS | 37° C. | 5% |
| Sawano | MEM/15% FBS | 37° C. | 5% |
| TCS | MEM/10% FBS | 37° C. | 5% |
| UtSMC | SmBM/5% FBS | 37° C. | 5% |
| HepG2 | DMEM/10% FBS | 37° C. | 5% |
| HC | CS-C/10% FBS | 37° C. | 5% |
| A549 | DMEM/10% FBS | 37° C. | 5% |
| MRC-5 | HF-RTC80-7/10% FBS | 37° C. | 5% |
| Caco-2 | MEM/20% FBS + | 37° C. | 5% |
| | 1% NEAA | | |
| Vero | MEM/10% FBS | 37° C. | 5% |
| COS-7 | DMEM/10% FBS | 37° C. | 5% |
| NIH3T3 | DMEM/10% CS | 37° C. | 5% |

TABLE 4
Cytocidal effect of the protein purified from BT strain
A1470 on various cell lines

| Origin | cell line | tissue | cell type | EC50 (μg/ml) |
|---|---|---|---|---|
| human | MOLT-4 | blood | T cell leukemia | 0.42 |
| | Jurkat | blood | T cell leukemia | 0.25 |
| | HL-60 | blood | myeloblast cell | 0.17 |
| | HeLa | cervical | cervical cancer | >10 |
| | Sawano | uterus | cervical cancer | <0.07 |
| | TCS | cervical | cervical cancer | 3.2 |
| | UtSMC | cervical | cervical smooth muscle | >10 |
| | HepG2 | liver | hepatoma cell | 0.16 |
| | HPC | liver | normal liver cell | >10 |
| | A549 | lung | embryonic fibroblast | >10 |
| | MRC-5 | lung | cancer cell | >10 |
| | Caco-2 | colon | adenocarcinoma | 0.57 |
| monkey | Vero | kidney | cancer cell | >10 |
| | COS-7 | kidney | SV40 transformed cell | >10 |
| mouse | NIH3T3 | embryo | fibroblast | 0.3 |

The results shown in Table 4 indicate that the 28 kDa protein of the present invention specifically recognized certain cells. It is also indicated that the protein of the present invention showed a cytopathic activity to the recognized cells.

The present invention enables research and development of proteins having a cell-recognizing activity and/or a cytopathic activity at gene level and molecular level. The present invention also enables establishment of a mass production technique of proteins. Therefore, the present invention is considered to contribute vastly to the techniques in a wide range of fields, such as development of DDS technique in the pharmaceutical field, development of diagnostics, development of certain cells in the agriculture and fishery field and breeding of animals and plants by selective destruction of tissue, as well as development of novel agents for controlling pest in chemical industry field. Furthermore, the protein of the present invention is useful for specifically recognizing and/or killing certain cells, based on the high selectivity it has. In addition, the protein of the present invention is useful as a pharmaceutical composition (specifically anticancer agent and cell-recognizing agent), because of the high specificity it has for certain cancer cells.

Free Text of Sequence Listing

SEQ ID NO: 4: A primer for amplifying a gene represented by SEQ ID NO: 1.

SEQ ID NO: 6: A primer for amplifying a gene represented by SEQ ID NO: 1.

1. An isolated nucleic acid molecule consisting of the nucleotide sequence represented by SEQ ID NO: 1.

2. An isolated nucleic acid molecule encoding a protein consisting of the amino acid sequence represented by SEQ ID NO: 2.

3. An isolated nucleic acid molecule which hybridizes under stringent condition to a nucleic acid molecule having a nucleotide sequence complementary to the nucleotide sequence represented by SEQ ID NO: 1, and which encodes a protein having a cell-recognizing activity in an activated form.

4. The nucleic acid molecule of claim 3, wherein the activated form is substantially free of a haemolytic activity.

5. The nucleic acid molecule of claim 3, wherein a cell-recognizing activity is specific to at least one cell selected from the group consisting of a leukemia cell, a hepatoma cell, a uterus cancer cell and a large bowel cell.

6. The nucleic acid molecule of claim 3, wherein the nucleic acid molecule is derived from geneus Bacillus.

7. An isolated nucleic acid molecule which hybridizes under stringent condition to a nucleic acid molecule having a nucleotide sequence complementary to the nucleotide sequence represented by SEQ ID NO: 1, and which encodes a protein having a cytopathic activity in an activated form.

8. The nucleic acid molecule of claim 7, wherein the activated form is substantially free of a haemolytic activity.

9. The nucleic acid molecule of claim 7, wherein the cytotixic activity is specific to at least one cell selected from the group consisting of a leukemia cell, a hepatoma cell, a uterus cancer cell and a large bowel cell.

10. The nucleic acid molecule of claim 7, wherein the nucleic acid molecule is derived from geneus Bacillus.

11. A vector comprising the nucleic acid molecule of any of claims 1, 3 and 7.

12. A transformant comprising the vector of claim 11.

13. An isolated protein consisting of the amino acid sequence represented by SEQ ID NO: 2.

14. An isolated protein comprising an amino acid sequence wherein 1 or more amino acids are deleted, substituted, added or inserted in the amino acid sequence represented by SEQ ID NO: 2, whose activated form has a cell-recognizing activity.

15. The protein of claim 14, whose activated form is substantially free of a haemolytic activity.

16. The protein of claim 14, wherein the cell-recognizing activity is specific to at least one cell selected from the group consisting of a leukemia cell, a hepatoma cell, a uterus cancer cell and a large bowel cell.

17. The protein of claim 14, wherein the protein is derived from genus Bacillus.

18. An isolated protein comprising an amino acid sequence wherein 1 or more amino acids are deleted, substituted, added or inserted in the amino acid sequence represented by SEQ ID NO: 2, whose activated form has a cytopathic activity.

19. The protein of claim 18, whose activated form is substantially free of a haemolytic activity.

20. The protein of claim 18, wherein the cytopathic activity is specific to at least one cell selected from the group consisting of a leukemia cell, a hepatoma cell, a uterus cancer cell and a large bowel cell.

21. The protein of claim 18, wherein the protein is derived from genus Bacillus.

22. An isolated protein, which is obtained by solubilizing the protein of any of claim 13, 14 and 18 under alkaline conditions, followed by protease treatment.

23. A pharmaceutical composition comprising the activated form of the protein of claim 13 or 14.

24. A cell-recognizing agent comprising the activated form of the protein of claim 13 or 14.

25. An anticancer agent comprising the activated form of the protein of claim 13 or 18.

26. A method for producing the protein of any of claim 13, 14 and 18, comprising the steps of:
(a) culturing a cell expressing said protein in a culture medium; and
(b) recovering said protein.

27. An antibody directed to the protein of any of claims 13, 14 and 18.

28. A method for identifying a cell recognized by-the activated form of the protein of claim 13 or 14, comprising the steps of:
(a) incubating the activated form of the protein with any cell in a culture medium; and
(b) determining whether the activated form of the protein shows a cell-recognizing activity to said cell.

29. A method for identifying a cell to which the activated form of the protein of claim 13 or 18 shows a cytopathic activity, comprising the steps of:
(a) incubating the activated form of the protein with any cell in a culture medium; and
(b) determining whether the activated form of the protein shows a cytopathic activity to said cell.

30. A method for identifying a substance having an affinity for the activated form of the protein of claim 13 or 14, comprising the steps of:
(a) contacting the activated form of the protein with an analyte; and
(b) determining whether said analyte has an affinity for the activated form of the protein.


SEQUENCE LISTING (6 sequences)

SEQ ID NO: 1 - DNA, 837 bp, Bacillus thuringiensis
atggccattattaatcttttgcgagaattagaaatatacggaatgcagtatgcgaatagccaccagtatacgtatggttcaagctattcagatgatacgaatccaattcgaatagcaggtttagatgcgcgcattccagatccgattgtgacagatcctgtgaatcatattgtgttagatcgaagaatcattacgaatacgacttctaattcattagaaggtgtattcagcttttcgaatgcgtatacgagtcgaacatcctcacaaaccagagatggtgtaacagcgggaacaaatatcaccggtaaatattttgcaaatttattttttgagcaagtaggtttatcaggtagaatagcttttgaaggagccgttacaaatgagaacaaatatacgttagacgcgacccaagattttagagattcacagacaatacgtgtgccgcctttccatcgagcgacaggtgtatacacattagaacagggagcattcgaaaaaatgactgttttagaatgtgtggtatccggaaatgggattatcagatattatcgaacgcttcctgataatagttatacagaaatcgttcaacgcgtgaatataatagatgtgttgcaagcaaatggaacgcctggctttacgatatcaaaggaacaaaatagggcgtactttacaggtgaaggaacgatatcaggtcaaataggcctgcaaacatttatagatgtagtaatcgagccgttaccaggtcatgccggacaaacgcaaaagtaccaaattccggttacgggacaaagtggattagatattcctatttttgactcggtaggatatcgacaataa

SEQ ID NO: 2 - protein, 278 aa, Bacillus thuringiensis
MAIINLLRELEIYGMQYANSHQYTYGSSYSDDTNPIRIAGLDARIPDPIVTDPVNHIVLDRRIITNTTSNSLEGVFSFSNAYTSRTSSQTRDGVTAGTNITGKYFANLFFEQVGLSGRIAFEGAVTNENKYTLDATQDFRDSQTIRVPPFHRATGVYTLEQGAFEKMTVLECVVSGNGIIRYYRTLPDNSYTEIVQRVNIIDVLQANGTPGFTISKEQNRAYFTGEGTISGQIGLQTFIDVVIEPLPGHAGQTQKYQIPVTGQSGLDIPIFDSVGYRQ

SEQ ID NO: 3 - protein, 18 aa, Bacillus thuringiensis
AIINLLRELEIYGMQYAN

SEQ ID NO: 4 - DNA, 20 bp, Artificial Sequence
  PCR primer for amplifying gene derived from Bacillus thuringiensis A1470
gaaatwtatggwatgcaata

SEQ ID NO: 5 - protein, 19 aa, Bacillus thuringiensis
GAVTNENKYTLDATQDFRD

SEQ ID NO: 6 - DNA, 20 bp, Artificial Sequence
  PCR primer for amplifying gene derived from Bacillus thuringiensis A1470
gtatatttattttcattwgt